(19) United States
(12) Patent Application Publication
(10) Pub. No.: US 2011/0078805 A1
(43) Pub. Date: Mar. 31, 2011

(54) COMPOSITIONS AND METHODS FOR GENERATING TRANSGENIC ANIMALS

(75) Inventors: Lisa S.D. EMMETT, Luthersburg, PA (US); Theresa GRATSCH, Ann Arbor, MI (US); K. Sue O'SHEA, Ann Arbor, MI (US); J. Matthew VELKEY, Ann Arbor, MI (US); Michael J. WELSH, Dexter, MI (US); William WU, Ann Arbor, MI (US)

(73) Assignee: The Regents of The University of Michigan, Ann Arbor, MI (US)

(21) Appl. No.: 12/841,010

(22) Filed: Jul. 21, 2010

Related U.S. Application Data
(63) Continuation of application No. 11/939,434, filed on Nov. 13, 2007.
Application No. 11/939,434 is a continuation of application No. 11/728,138, filed on Mar. 23, 2007, now abandoned.
(60) Provisional application No. 60/785,316, filed on Mar. 23, 2006.
(60) Provisional application No. 60/876,719, filed on Dec. 22, 2006.
(60) Provisional application No. 60/900,185, filed on Feb. 8, 2007.

(51) Int. Cl. A01K 67/027; A01K 67/02; C12N 5/10

(52) U.S. Cl. 800/13; 800/21; 435/325

(57) ABSTRACT

The present invention provides methods of altering gene expression of embryos to provide compositions and methods for efficient generation of transgenic animals. In particular, the present invention provides compositions and methods for generating germ-line transgenic animals by direct injection of nucleic acid molecules into animals.

### CROSS-REFERENCE TO RELATED APPLICATIONS

This application is a continuation of U.S. application Ser. No. 11/939,434, filed Nov. 13, 2007, which is itself a continuation of U.S. application Ser. No. 11/728,138, filed Mar. 23, 2007, which claims priority to U.S. Provisional Patent Application Ser. Nos. 60/785,316, filed Mar. 23, 2006, 60/876,719, filed Dec. 22, 2006, and 60/900,185, filed Feb. 8, 2007, each of which is herein incorporated by reference in its entirety.

### STATEMENT REGARDING FEDERALLY SPONSORED RESEARCH OR DEVELOPMENT

The present invention was made with government support under ES-011188, NS-039438, and RR-023187 awarded by the National Institutes of Health. The government has certain rights in this invention.

### FIELD OF THE INVENTION

The present invention provides methods of altering gene expression of embryos to provide compositions and methods for efficient generation of transgenic animals. In particular, the present invention provides compositions and methods for generating germ-line transgenic animals by direct injection of nucleic acid molecules into animals.

### BACKGROUND

Transgenic animals are commonly generated via microinjection of DNA or genes of interest into fertilized oocytes, by transplantation of a stably transfected somatic cell nucleus into an enucleated oocyte, or by stable transfection of embryonic stem (ES) cells and injection of ES cells into blastocyst-embryos followed by reimplantation of injected oocytes, nucleus-transplanted oocytes, or injected blastocyst embryos into the uterus of a pseudopregnant recipient female. Such procedures are very complicated, time-consuming, and costly.

What are needed are easier, more efficient, and less costly methods for making transgenic animals. Additionally, some animal types are not amenable to transgenic modification using existing methods. Thus, new methods are needed that permit the generation of a range of different transgenic animal types.

### SUMMARY OF THE INVENTION

The present invention provides compositions and methods for efficient generation of transgenic animals. In particular, the present invention provides compositions and methods for generating germ-line transgenic animals by direct injection of nucleic acid molecules into animals.

In some embodiments, the present invention provides simple, rapid, and low cost methods for generating transgenic animals. For example, in some embodiments, DNA to be transfected is administered to an animal via intravenous injection. Although intravenous delivery of DNA is widely used for gene expression and regulation in animals, generation of germ-line (e.g., episomal and/or chromosomal) transgenics has not been reported. Indeed, previous studies indicated that intravenous delivery of DNA into pregnant mice could not result in germ line transgenics. In contrast, the compositions and methods of the present invention provide germ line transmission and permit the generation of offspring (e.g., F1, F2, etc.) that carry and express the transgene of interest.

In some embodiments, the DNA of interest is administered without the use of transfection reagent carriers, although the present invention is not so limited. This is in contrast to methods that employ liposomes or other carriers. In some embodiments, the DNA of interest is administered with an appropriate nucleic acid carrier, for example, liposomes, DEAE-dextran, cationic lipids, calcium phosphate or other carriers known to those skilled in the art of DNA transfection.

In some embodiments, the DNA is administered intravenously via the tail vein of the subject animal, although the present invention is not so limited. In some embodiments the DNA administration may involve injection into another vein (e.g., not the tail vein), an artery, or into tissue adjacent to a vein, an artery, capillaries or lymph nodes. In some embodiments the administration is via a technique as described in U.S. Provisional Patent Application, 60/900,185, filed Feb. 8, 2007, or O'Shea et al., J. Biomed. Biotechnol., 18657 (2006), herein incorporated by reference in their entireties.

In some embodiments, the DNA is administered to pregnant subjects such that their offspring that have the transgene can pass the transgene to the next generation. In some embodiments, the pregnant subjects are at an early stage of pregnancy.

The present invention is not limited by the nature of the animal. In some embodiments, the animal is a rodent (e.g., mouse, rat, chipmunk, prairie dog, squirrel, beaver, gopher, hamsters, voles, gerbils, porcupines, guinea pigs, etc.). In some embodiments, the animal is a livestock animal (e.g., pigs, cattle, goats, deer, sheep, yaks, etc.). In some embodiments, the animal is a companion animal (e.g., cat, dog, etc.). In some embodiments, the animal is a primate (e.g., lemurs, monkeys, apes, humans, etc.). In some embodiments, the animal is a marsupial (e.g., kangaroos, opposums, possums, wombats, bettongs, moles, etc.). In some embodiments, the animal is a non-human animal (e.g., non-human mammal).

The present invention is also not limited by the nature of the DNA that is provided. In some embodiments, the DNA is in the form of a plasmid, a linear construct (e.g., a linear construct with even ends, uneven ends, or modified ends such as dumbbell ends (Wooddell et al., 2005, Biochem. Biophys. Res. Comm. 334:117, herein incorporated by reference in its entirety)), an expression vector, a viral sequence (e.g., adenoviral sequence, lentiviral sequence), etc. In some embodiments, the DNA comprises gene expression enhancer sequences, insulator sequences, scaffold/matrix attachment regions (S/MARs) (see e.g., Conese et al., Gene Ther., 11:1735 (2004) and Tetko et al., Plos Comput. Biol. 2:e21 (2006), herein incorporated by reference in their entireties), etc, whether natural or artificial, as well as any sequences that are functionally equivalent. In some embodiments, the DNA encodes a gene of interest (e.g., a marker gene, a therapeutic gene, an siRNA, a protein or peptide fragment, an miRNA, an shRNA, an immunoglobulin, etc.). In some embodiments, multiple different transgenes are provided (e.g., in one or more different vectors). In some embodiments, the DNA comprises a recombination sequence (e.g., a sequence that permits homologous recombination into the genomic DNA). For example, in some embodiments, the DNA comprises one or more sequences that permit recombination via Cre/lox systems, flp recombinase systems, or other recombinase systems.

In some embodiments, the present invention provides methods for generating transgenic offspring comprising: injecting a pregnant animal with DNA under conditions such that the offspring are germ-line transgenic for the DNA. In some embodiments, the DNA is injected intravenously into the pregnant animal (e.g., in the tail vein). In some embodiments, the offspring express the DNA of interest. In some embodiments, the offspring can transmit the transgene to their offspring (i.e., the F1 generation). In some embodiments, the F1 generation can transmit the transgene to their offspring (i.e., the F2 generation). It is contemplated that the F3, F4, F5, etc. generations may also be generated. The present invention also provides transgenic animals generated by the methods (e.g., chimeric, transgenic animals).

In some embodiments, the methods comprise one or more of the steps of: injecting DNA and reagents into pregnant animals; propagating embryos from the injected pregnant animals; deliver offspring (F0 generation; founders) from pregnant animals; growing the F0 animals to mature stage; breeding the matured F0 animals; propagating embryos from pregnant F0 animals; giving birth to offspring (F1 generation) by the pregnant F0 animals; growing the F1 generation to mature animals; breeding males and females between the matured F1 animals or between F0 and F1 animals; and producing next generations of animals (e.g., F2, F3, F4, . . .).

In some embodiments, methods are carried out to confirm gene transfer. In some such embodiments, the method comprises extracting genomic DNA and verifying genome integration of the injected DNA (e.g., via PCR (e.g., inverse PCR), Southern blot, DNA sequencing, chromosomal staining, etc.). In some embodiments, the method comprises isolating RNA and assaying for mRNA expressed from the integrated DNA. In some embodiments, a phenotypic method is conducted. For example, in some embodiments, proteins are extracted to assay for the expression of the integrated DNA. In other embodiments, phenotypic changes in the animal are observed (e.g., color production, size, weight, presence or absence of disease state, other traits, etc.).

Injections may be made using any suitable mechanism. In some embodiments, a needle is employed. In some embodiments, injections are conducted using needleless systems, such as compressed air injection. In some embodiments, nucleic acid is affixed to a particle (e.g., lipid complex, metal particle, dendrimer, etc.) and the particle/nucleic acid complex is administered. Any means for administration, now known, or later developed, is contemplated for use in embodiments of the methods of the present invention.

### DEFINITIONS

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include tissues and blood products, such as plasma, serum and the like. Such examples are not however to be construed as limiting the sample types applicable to the present invention.

As used herein, the term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms.

As used herein, the terms “variant” and “mutant” when used in reference to a nucleic acid or polypeptide refer to a nucleic acid sequence or an amino acid sequence that differs by one or more nucleic acids or amino acids from another, usually related nucleic acid or polypeptide. The variant may have “conservative” changes, wherein a substituted nucleic acid encodes for the same amino acid, or an amino acid has similar structural or chemical properties. The variant may have “non-conservative” changes (e.g., replacement of a nucleic acid that changes an amino acid). Similar minor variations may also include nucleic acid or amino acid deletions (e.g., truncations) or insertions (in other words, additions), or both. Guidance in determining which and how many nucleic acids or amino acid residues may be substituted, inserted or deleted without abolishing biological activity or causing a desired abolishment of activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

As used herein, the term “gene” refers to a nucleic acid (e.g., DNA or RNA) sequence that comprises coding sequences necessary for the production of an RNA, or a polypeptide or its precursor (e.g., a propeptide or xymogen). A functional polypeptide can be encoded by a full-length coding sequence or by any portion of the coding sequence as long as the desired activity or functional properties (e.g., fluorescent properties, enzymatic activity, antibiotic, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene. A gene also encompasses the coding regions of a structural gene and includes sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences teemed “introns” or “intervening regions” or “intervening sequences.” Introns are segments of a gene that are transcribed into hetero-nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. Genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

As used herein, the term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise a gene sequence that comprises cDNA forms of the gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

As used herein, the term “polynucleotide” refers to a molecule comprised of two or more deoxyribonucleotides or ribonucleotides, preferably more than three, and usually more than ten. The exact size will depend on many factors, which in turn depends on the ultimate function or use of the polynucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

As used herein, the term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below. Nucleic acids can be either deoxyribonucleic acids (DNA, e.g., genomic DNA, cDNA, plasmid DNA, etc.) or ribonucleic acids (RNA, e.g., siRNA, miRNA, shRNA, mRNA, dsRNA, etc.).

As used herein, the term “region” or “portion” when used in reference to a nucleic acid molecule refers to a set of linked nucleotides that is less than the entire length of the molecule.

As used herein, the term “a nucleic acid sequence encoding” a specified DNA or RNA molecule or polypeptide refers to a nucleic acid sequence comprising the coding region of a gene or in other words the nucleic acid sequence that encodes a gene product. The coding region may be present in either cDNA, genomic DNA or RNA foam. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

As used herein, the term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

As used herein, the terms “complementary” and “complementarity” refer to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “homology” when used in relation to nucleic acids or amino acids refers to a degree of similarity or relatedness, as for example between base sequences in different nucleic acid sequences, or between base sequences in different regions of a nucleic acid sequence. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the term “vector” refers to nucleic acid molecules that transfer DNA segment(s) from one cell to another. A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non-transfected cells, or reporter proteins, by means of which an effect on expression or activity or function of the reporter protein can be monitored.

As used herein, the term “expression cassette” refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals

As used herein, the term “expression vector” refers to a vector comprising one or more expression cassettes.

As used herein, the term “transgene” refers to a heterologous gene that is transferred or placed into an organism.

As used herein, the phrase “germ-line transgenic” refers to an animal that contains a transgene, wherein the transgene is present in the germ cells of the animal such that the transgene can be passed to the offspring of the animal through reproduction. Germ-line transgenic animals can be identified, for example, by observing offspring for the presence of the transgene or expression of the transgene. Germ-line transgenic animals can also be identified, for example, by examining the germ cells of the transgenic animal for the presence of a transgene, incorporated in a manner consistent with heritability.

As used herein, the term “selectable marker” or “marker gene” refers to a gene which encodes an enzyme having an activity that confers resistance to an antibiotic or drug upon the cell in which the selectable marker is expressed, or which confers expression of a trait which can be detected (e.g.., luminescence or fluorescence). Selectable markers may be “positive” or “negative.” Examples of positive selectable markers include the neomycin gene that confers resistance to G418 and to kanamycin, and the bacterial ampicillin resistance gene (amp), which confers resistance to the antibiotic ampicillin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat Nos., 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif., references of which are incorporated herein in their entireties), chloramphenicol acetyltransferase, β-galactosidase, alkaline phosphatase, and horse radish peroxidase. A variety of other reporter genes are known. Currently known, as well as future developed reporters, find use in embodiments of the present invention.

As used herein, the term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring. A wild-type gene is often designated as the gene that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product. For example, many naturally occurring sequences contain single nucleotide polymorphisms (SNPs) that differ from wild type sequences.

As used herein, he term “transfection” refers to the introduction of foreign DNA into cells.

As used herein, the term “siRNA” refers to short interfering RNAs. In some embodiments, siRNAs comprise a duplex, or double-stranded region, where each strand of the double stranded region is about 18 to about 25 nucleotides long; the double stranded region can be as short as 16, and as long as 29, base pairs long, where the length is determined by the antisense strand, although both longer and shorter lengths may be used. Often siRNAs contain from about two to four unpaired nucleotides at the 3′ end of each strand. siRNAs appear to function as key intermediaries in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

As used herein, the teen “RNA interference” or “RNAi” refers to the silencing or decreasing expression, or inhibition of expression, of gene expression by siRNAs. It is the process of sequence-specific, post-transcriptional gene silencing in animals and plants, initiated by siRNA that is homologous in its duplex region to the sequence of the silenced gene or that is complementary in its duplex region to the transcriptional product of the silenced gene. The gene may be endogenous or exogenous to the organism, present integrated into a chromosome or present in a transfection vector that is not integrated into the genome. The expression of the silenced gene is either completely or partially inhibited.

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 30% free, at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

As used herein, the term “subject” refers to any animal (e.g., a mammal), including, but not limited to, humans, non-human primates, rodents, and the like, which is to be the recipient of a particular treatment.

As used herein, the term “animal” refers to any animal (e.g., a mammal), including, but not limited to, humans, non-human primates, rodents (e.g., mice, rats, etc.), and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, etc. Non-human animals can equally be oviparous animals including, but not limited to, chickens, ducks and geese.

### DETAILED DESCRIPTION OF THE INVENTION

The following description provides exemplary embodiments of the present invention. The present invention is not limited to these particular embodiments.

It was discovered during the development of embodiments of the present invention that when genetic or nucleic acid material is injected into the pregnant mother at the right time and in the right conditions, the genetic or nucleic acid material is expressed in the cells of embryo, including the primordial germ cells, and the offspring are transgenic and are able to pass the transgene to later generations.

As will be explained in greater detail later, the method of altering the genes of the embryo in the uterus is done, in some embodiments, by a process comprising the steps of:

A: preparing a pregnant animal carrying the embryo for at least one injection,

B: inserting a hollow device into a blood transport vessel of the pregnant animal, and

C: introducing genetic or nucleic acid material into the blood transport vessel through the hollow device.

When using the technique as detailed in this specification, the genetic or nucleic acid material was unexpectedly passed through the placenta, a known barrier to various materials and the genetic or nucleic acid material was unexpectedly taken up and expressed (present) in all the cells of the embryo. Once present in all the cells, the effect of the alteration can be studied upon the embryo, the embryo's development, the animal after it is born, and the animal's offspring.

The experiments supporting this disclosure have shown that the transgene, or altered gene, is present in every cell of the transduced embryos and in the primordial germ cells that form sperm and eggs. This offers the ability to address genetic defects, or dim (decrease expression of) disease genes or to add missing genes or increase the expression of gene, to model diseases, and to discover how genes function in both development and in birth defects.

The present invention also provides compositions and methods for efficient generation of transgenic animals. In particular, the present invention provides compositions and methods for generating germ-line transgenic animals by direct injection of nucleic acid molecules into animals. Certain specific embodiments of the present invention are described below to illustrate various features of the invention. The present invention is not limited to these particular embodiments.

The present invention is not limited to a particular mechanism. Indeed, an understanding of the mechanism is not necessary to practice the present invention. Nonetheless, it is contemplated that the methods of the present invention depend on the stage of development of the embryo, particularly on the characteristics of the early germ-line cells (e.g., primordial germ cells or gonocytes) that allow uptake of nucleic acid molecules. It is contemplated that for placental mammals this is the time when the germ-line cells are still associated with the yolk sac, or when these germ-line cells are migrating to, or have recently completed migration to, the genital ridges, or are rapidly proliferating in the developing gonads. It is also contemplated that this method is applicable to animals that lay eggs (i.e., oviparous animals), for example, if the nucleic acid molecules were injected into the eggs, or the embryonic circulation, at a time of embryonic development similar to that in which this method is effective for mammals.

The effectiveness of the technique to alter the genes of the embryo is detailed in the Experimental Section. The experiments demonstrate that the technique reliably expresses the genetic or nucleic acid material of interest in the embryo.

As is evident to one of skill in the art, being able to target the genes in the developing embryo by injecting the genetic or nucleic acid material of interest into the pregnant mother offers many opportunities. First, for many therapies, genes will need to be dimmed, not removed. One example is huntingtin, the gene that is mutated in Huntington's disease.

As huntingtin is expressed in every cell, it presumably has a function that has yet to be identified. Therefore, one would want to dim the expression of huntingtin, but not eliminate it entirely. By reducing its expression however, one could affect a therapeutic level that would not affect the cells in regions other than the brain. The invention also provides the ability to control gene expression in a tissue-specific manner by, for example, using tissue-specific promoter sequences that drive the expression of a transgene or by inserting promoter sequences or other sequences that regulate expression in operable combination with an endogenous gene.

Many birth defects are caused by what is called “gain of function,” a mutation that increases the expression or activity of a protein. By injecting the genetic or nucleic acid material targeting that mutation into the pregnant mother, genetic alterations causing birth defects could be easily targeted prenatally (before tissue damage is done).

The techniques disclosed in this specification have been demonstrated to target multiple genes using a single plasmid. Thus, in the case of Parkinson's disease, this technique could enable one to target, for example, both the precursor protein and the enzyme that degrades dopamine.

The technique can also be used to target multiple regions of a gene. This variation of the genetic or nucleic acid material that is injected can be used when one is not sure which region of the gene is the most important.

The technique can be used to target multiple genes in a signaling pathway to understand their role in development or disease.

Researchers can also limit the alterations in the embryo by targeting genes in a particular tissue (e.g. the brain) by using a promoter that is expressed only in the nervous system. This technique could then be used to only treat the brain effects of diseases like Alzheimer's, while not affecting other tissues of the body.

This technique can also be used to more rapidly determine the therapeutic amount or length of treatments. For instance, developing constructs that can be turned on and off may be useful for the treatment of Parkinson's. For example, in the case of the fetal tissue implants in Parkinson's patients, they may make too much dopamine and cause side effects. By having controlled expression, one can monitor this and achieve a therapeutic level. Such controlled expression is rapidly obtained by using the technique disclosed in this specification.

Experiments conducted during the development of the present invention demonstrated that early pregnant animals could be injected via tail vein with plasmid DNA constructs containing a reporter gene GFP (Green Fluorescent Protein) and an ampicillin resistance gene. The male and female offspring (F0 generation) from the injected females were allowed to breed to generate an F1 generation. Using PCR of genomic DNA, it was found that greater than 30% of the F0 generation of animals bore

DNA containing sequences for GFP and ampicillin resistant genes that were injected into parent animals. F1 generation animals also demonstrated GFP and ampicillin DNA in their DNA and in all F1 generation animals tested that were positive for GFP and ampr sequences in their DNA. GFP mRNA expression was demonstrated by RT-PCR. GFP protein expression was also demonstrated by western blot of F1 generation animals. This data demonstrates that the GFP and ampicillin genes were passed from parent into F0 and were transferred from F0 to F1 generation, producing animals that contained the transgene in their germ lines.

Thus, the present invention also provides compositions and methods for the generation of transgenic animals, including animals that incorporate one or more transgenes into germ-line cells. Such animals find use for numerous purposes, including biological research, including the development of model animals of disease and other biomedical/pharmaceutical applications. Methods include, but are not limited to, gene expression analysis; regulation and functional assays (overexpression, underexpression, knock-down expression, gene knock-out, gene knock-in, targeting); production of recombinant proteins, peptides, antibodies, etc.; etc.

The present invention provides methods for generation of transgenics for a wide variety of animal species, including animals that have historically proven difficult or impossible to generate using available techniques (e.g., oocyte injection, or somatic cell nuclear transfer, or ES cell methods). For example, the present invention provides transgenic guinea pigs generated by the methods of the present invention. Other valuable animals include transgenic mini-pigs for drug research. The present invention is not limited by the animal employed and can include, for example, non-human animals and oviparous animals as defined herein.

In some embodiments, the DNA is administered to the pregnant subject during early gestation, although the present invention is not so limited. A preferred time, for appropriate animal species, is after implantation of the embryo into a uterine wall and prior to establishment of the fetal circulation. Another preferred time for introduction is the time the embryo begins gastrulation and prior to establishment of fetal circulation. This corresponds to the second half of the first trimester when the placental barrier is most permeable.

For example, with mice having an average gestation period of 19-21 days, the present invention contemplates administering the DNA at time between, for example, days 1-18. In some embodiments, the DNA is administered during the first 10% time period of gestation (i.e., for a 20 day gestation period, during the first 2 days), during the first 20%, 30%, 40%, 50%, 60%, etc. time period of gestation. In some embodiments, the DNA is administered between the 10% and 20% (i.e., between days 2 and 4 for a 20 day gestation period), between the 10% and 30%, between the 10% and 40%, etc., between the 20% and 30% (i.e., between days 6 and 8 for a 20 day gestation period), between the 20% and 40%, etc. time periods. All intervals are also contemplated (e.g., treating female mice between days 6-6.5 of pregnancy).

Because of the ease of use of the methods, speed, and low cost, the present invention provides methods for large-scale generation of transgenic animals that would not otherwise be feasible. Routine generation of animals may be employed by an expanded range of researchers and practitioners.

In one embodiment, the present invention contemplates methods of use of animals where DNA has been incorporated into the germ line as previously described. These animals will find utility as, for example, in vivo experimental reservoirs for test compound screening. For example, the efficacy of a compound, agent, or drug is administered to an animal created to express a particular protein using the method of the present invention. If the compound, agent, or drug has an impact on protein expression, activity, or function, then that animal can be used as a tool for screening compounds, agents, or drugs that affect that protein. If the transgenic gene that codes for an oncogene (e.g., cancer causing gene) is transferred via the germ line of an animal using the method of the present invention, then compounds that inhibit the expressed oncogene are screened for in this transgenic animal. In some embodiments, an animal created by the method of the present invention no longer contains a gene (e.g., a knockout genotype). For example, the method of the present invention is used to create an animal that lacks a sodium channel, or a sodium channel subunit (e.g., α, β, γ, or δ sodium channel subunits). These animals can then be used as tools to study the effect of such a channel deletion on a mammalian system. Conversely, an animal created using the method of the present invention can overexpress a protein or underexpress a protein. This type of animal can be used to study the effect of such over or under expression on a mammalian system, as well as a tool to screen for compounds that inhibit protein expression or induce protein expression (e.g., siRNA), respectively.

In some embodiments, the transgene injected into the animal is a reporter gene (e.g., GFP, red fluorescent protein, tyrosinase, luciferase, chloramphenical acetyltransferase, etc.). In some embodiments, the transgene is a selectable marker gene (e.g., neomycin, ampicillin, hygromycin, bleomycin, etc.). In some embodiments, the transgene is a gene associated with a particular cancer (e.g., an oncogene, BRCA1, BRCA2, etc.). In some embodiments, the transgene encodes for a receptor and/or its ligand (e.g., MHC factor receptors and their ligands, etc.). In some embodiments, the transgene encodes for a viral protein (e.g., HIV proteins, HSV proteins, etc.). Those skilled in the art will appreciate the myriad (or multitude) of transgenes that can be utilized by the method of the present invention.

In one embodiment, the present invention contemplates the injection of DNA into the tail vein of a rodent (e.g., rat, mouse, prairie dog, etc.). In some embodiments, the DNA is not associated with a carrier. In some embodiments, the DNA being injected is linear DNA. In some embodiments, the DNA being injected is non-linear plasmid DNA. In some embodiments, the DNA being injected contains sequences that block enhancer-promoter interactions and protect the transgene from chromosomal positional effects (e.g., insulator or blocker sequences). For example, insulator sequences include, but are not limited to chicken β-globin (Recillas-Targa F et al., 1999, Proc. Natl. Acad. Sci. 96:14354-9), and insulators described and contemplated in Drebs J E and Dunaway M, 1998 Mol Cell. 1:301-8, Parnell TJ and Geyer P K, 2000, EMBO J. 19:5864-5874, Cook PR, 2003, J. Cell Sci. 116:4483-91, Brasset E and Vaury C, 2005, Heredity 94:571-6, Wei, G H et al., 2005, Cell Res. 15:292-300, and Zhao H and Dean A, 2005, Biochem. Cell Res. 83:516-24, all references incorporated herein in their entireties.

The present invention is not limited to the dose of DNA provided to the subject. One skilled in the art will appreciate a range of appropriate doses based on the characteristics of the subject (e.g., weight, species, etc.). In some embodiments, from 100 nanograms to 1 mg of DNA is injected, although higher or lower doses may be used. In some embodiments, at least 0.2 micrograms of DNA is injected. In some embodiments, multiple doses are provided at two or more time points.

The present invention also provides kits for carrying out the methods of the present invention. In some embodiments, the kits comprise reagents useful, sufficient, or necessary for carrying the methods of the present invention. Such reagents include, but are not limited to, vectors, transgene sequences, control sequences, buffers, enhancer sequences, insulator sequences, scaffold/matrix attachment regions (S/MARs) sequences, reagents for use in detecting successful transfection, written instructions, software, and the like. The kits may also comprise components for carrying out an injection (e.g., a syringe, needle, antiseptic, etc.). In some embodiments, the kits comprise buffer solutions, liposomes or other nucleic acid delivery reagents, or other reagents or materials that affect the rate and location of uptake of nucleic acid molecules in the tissues of the embryo or its mother.

### EXPERIMENTATION

The following examples are provided in order to demonstrate and further illustrate certain preferred embodiments and aspects of the present invention and are not to be construed as limiting the scope thereof.

### EXAMPLE 1

### Exemplary Nucleic Acid Injection Technique

Method of altering the genes of the embryo in the uterus are conducted, in some embodiments, by a process comprising the steps of:

A: preparing a pregnant animal carrying the embryo for at least one injection,

B: inserting a hollow device into a blood transport vessel of the pregnant animal, and

C: introducing genetic material into the blood transport vessel through the hollow device.

The method is believed to be much more effective when the blood transport vessel has been dilated prior to the introduction of the genetic material, and preferably prior to the insertion of the hollow device. Dilating the blood transport vessel, in cases where the blood vessel is small, for example, can be achieved using techniques available to the practitioner which include the use of drugs or warming the pregnant animal. If drugs are used, care should be taken so as not to otherwise alter the development of the fetus. In some experiments, an alcohol swab over the tail produced vasodilation.

Warming the animal by exposing the animal to a temperature or combination of temperatures for a sufficient amount of time to dilate the blood vessel is a preferred method of dilating the blood vessel. In some embodiments, a warm blanket (approximately 10-20° C. above the temperature of the animal's body temperature) is used.

The animal is laid on the blanket for about 5-10 minutes, or until the extremities became pink. Dilation of the tail veins are visually observed at this time.

In some embodiments it is further advantageous to immobilize the blood transport vessel of the pregnant animal. Examples of immobilization of the blood transport vessel of the pregnant animal include anesthetizing the pregnant animal and sufficiently securing the pregnant animal to permit insertion of the hollow device into the blood transport vessel. The practitioner can have the pregnant animal itself immobilize the blood transport vessel (otherwise known as holding still). Animals that are cooperatively trainable may be able to do this. In humans, the practitioner could instruct the pregnant mother to hold still or not move the extremity containing the targeted blood transport vessel.

In the case of mice or their neonates, they may be placed into a small conical tube with a hole at both ends. The tail sticks out of one hole. While the tip of the tail can move, the tail vein at the base of tail is held virtually immobile so that the hollow device, in this case a needle, is inserted bevel up into the tail vein near the base of the tail.

While the hollow device used in these experiments may be a needle, with the preferred needle being a butterfly needle, a shunt, or other similar device could also be used to pass the genetic material into the blood transport vessel.

The size of the opening of the needle relative to the rate of delivery is important, in some embodiments, to the success of technique. Employing high-pressure teachings of earlier works (e.g. Lewis et al., Nature Genet., 32: 107 (2002)) can cause death of the mother. In contrast to the high pressure technique of Lewis et al., embodiments of the present invention employ a low pressure technique, with the DNA material or genetic material being introduced in a dilute solution over an extended period of time.

It is contemplated that any blood transport vessel (e.g., artery or vein), using this technique, can be used with the proviso that the artery or vein transports the blood from the point of insertion of hollow device, preferably a needle, to the embryo. Examples of suitable arteries and veins are: 1) uterine arteries that travel to the embryo; and 2) tail veins. In the case of animals without tails, such as humans, there are veins that are the functional equivalent of tail veins in mice. In some embodiments, in animals without tails, or with very small tails, other veins such as the saphenous vein may be used for injection. In some embodiments, for humans, a vein is used for both genetic transfer and for blood drawing for associated or unrelated medical procedures. In some embodiments, for human and other primates, the median cubital vein or the cephalic vein, or equivalents, is used.

The pressure at which the genetic material is introduced is called the “introduction pressure.” The introduction pressure is the pressure at the exit of the hollow device. The introduction pressure should be sufficiently low so as not to kill the pregnant animal.

Pressure at the exit side of the needle, or its functional equivalent should be less than the pressure that would kill the pregnant mother. In practical terms, this can be less than about the pressure required to pass 300 microliters of Ringer's solution at 23° C. through a 23 gauge needle in 10 seconds, or below about the pressure required to pass 300 microliters of Ringer's solution at 23° C. through a 23 gauge needle in 15 seconds or below about the pressure required to pass 300 microliters of Ringer's solution at 23° C. through a 23 gauge needle in 20 seconds or below about the pressure required to pass 300 microliters of Ringer's solution at 23° C. through a 23 gauge needle in 25 seconds, or below about the pressure required to pass 300 microliters of Ringer's solution at 23° C. through a 23 gauge needle in 30 seconds. Another variant is to introduce the genetic material through a drip into the blood transport vessel. This would be similar to an I.V. drip used to continuously administer drugs. Thus a catheter would work as the delivery device as well. In another embodiment, the introduction may be discontinuous, with the material being introduced on several occasions. If the pressure is too high, the mother will die.

If a needle is used, the pressure is usually generated by the plunger of a syringe. If a drip system like a catheter, the pressure is generated by keeping the storage device containing the genetic material above the hollow device entering the blood vessel.

Another method of introducing the genetic material is to use a carrier solution, with the genetic material preferably dissolved or suspended in the solution. The carrier solution should be of sufficient polarity to keep the genetic material unaggregated, as opposed to clumped or aggregated. The preferred distribution would be evenly distributed throughout the solution. Ringer's solution was used in the experiments and is an example of a suitable carrier.

### EXAMPLE 2

### Alteration of the Gene Expression in the Embryo

In a first set of experiments, the vector was DNA containing the promoter CMV—(cytomegalovirus) driving the expression of DsRed2 and a second promoter, U6, driving the expression of the hairpin DNA targeted to knockdown the expression of Bmp4 was injected into the tail vein of mice. In this set of experiments, all the cells of the embryos, fluoresced indicating that the construct was taken up and expressed in those cells. The chance of a false positive, where the marker is expressed, but the hairpin DNA is not, was eliminated by placing the marker and the DNA on the same backbone.

Because the hairpin DNA was on the same backbone as the marker and all the cells had the marker, there was no reason to believe that the Bmp4 had not been knocked down as well. This predictability has been confirmed over the course of 9 separate DNAs. In each case, using the disclosed technique, the marker was found in all cells of the embryo or neonate developed from the embryo. In all cases, the expression of the naked DNA introduced into the tail vein was individually confirmed at the protein and RNA level using immunohistochemistry and RT-PCR (reverse transcript, polymerase chain reaction).

Other experiments using the method described above under the conditions described show the effectiveness of the method to alter the gene expression of the embryo are Bmp4 (which, to the extent it can be analyzed, phenocopies the Bmp4 null embryos), Bmp7 alone, Bmp4 in combination with Bmp7, Wnt8a, Wnt8b (singly and in combination) (where Wnts are mammalian homologues of wingless genes identified in the fruitfly), Nanog, geminin, Est1, Est2, and Est3, where EST is an Expressed Sequence Tag (EST); which are genes (without names) that the inventors identified in a screen for genes whose expression in embryonic stem cells were unregulated by exposure to noggin; i.e., new genes.

In each case where an antibody is available to the protein (Bmp4, Nanog, geminin) or to the downstream signal transduction cascade (PhosphoSmad1,5,8) knock-down of the gene product was observed in individual embryos. In cases where an antibody is not available, success was demonstrated by observation of unique phenotypes, and knock-down by PCR (polymerase chain reaction).

The data demonstrates therefore that multiple targets can be knocked-down or reduced.—e.g., Bmp7 and Bmp4 and identify an additive phenotype.

Scrambled (missense) hairpin DNA exposed embryos were produced that resemble wild type and pRed embryos both in phenotype and gene expression profile. Multiple genes using a single construct (noggin, chordin and follistatin) and confined knock-down using quantitative PCR on individual embryos.

### EXAMPLE 3

### Injection and Expression of the Neomycin Gene in Pregnant Mice and their Offspring

Experiments were performed to test whether mice could be injected with an exogenous gene sequence, which would subsequently be expressed in downstream generations of offspring. Female mice, at day 6-6.5 of pregnancy, were injected in or near the tail vein with a plasmid expression vector encoding the neomycin antibiotic resistance gene.

A suitable administration protocol comprises dissolving 20 micrograms of vector DNA in sterile phosphate buffered saline (PBS) or physiological saline and injecting the solution into animals. A suitable vector concentration is 0.1 microgram per microliter (e.g., using 200 microliter volume to deliver 20 micrograms of vector). Injection rates may be slow, occurring over a period of 20 to 30 seconds. Both lower and higher doses may be used.

Reverse transcription polymerase chain reaction (RT-PCR) was utilized to analyze for the expression of neomycin mRNA in parent animals and their offspring, which on day 6.5 (injection date of expression vector) were egg cylinder stage embryos. Upon analysis with RT-PCR, it was determined that the neomycin mRNA was being expressed in most tissues of all parent animals injected, and most of F0 offspring animals.

Five pairs of offspring founder mice were subsequently bred, resulting in a second generation (F1) of 45 offspring. F1 offspring were tested for neomycin mRNA expression with RT-PCR. Of the 45 F1 animals tested, one tested positive for neomycin mRNA expression. To further verify that neomycin mRNA was present in this animal, a 300bp portion of the neomycin cDNA was amplified using polymerase chain reaction (PCR) and sequenced. Sequence analysis confirmed the presence of neomycin cDNA.

The results indicate that a cDNA construct can be delivered into the germ line of mouse embryos upon injection of the pregnant mother animal with plasmid DNA. The efficiency of 2% expression of the injected plasmid DNA into subsequent generations of offspring is comparable to that of pronuclear injection of oocytes and re-implantation of the injected oocytes into pseudopregnant recipient foster-mother mice.

### EXAMPLE 4

### Injection and Expression of GFP and Ampicillin Genes in Pregnant Mice and their Offspring

As in Example 3, female mice, at day 6-6.5 of pregnancy, were injected with a plasmid expression vector encoding both green fluorescent protein (GFP) and the amplicillin antibiotic resistance gene. Genomic DNA from the resulting offspring was analyzed for the presence of both GFP and ampicillin DNA using polymerase chain reaction (PCR). A total of 35 founder offspring (both male and female) from 5 litters were tested for the presence of GFP and ampicillin resistance (ampr) gene sequences in their DNA by PCR. Of these, one or more animals from each litter and a total of 12 animals were positive for both GFP and ampr sequences. The PCR products (520 bases for GFP and 534 bases for ampicillin resistance gene) were confirmed to be that of GFP and ampr by sequencing the PCR products.

Pairs of offspring animals were subsequently mated to produce second generation (F1) animals. Genomic DNA from F1 animals was analyzed by PCR for GFP and ampicillin gene sequences. F1 animals were found to have both GFP and ampicillin gene sequences in their genomic DNA as indicated by PCR, with the PCR products' identities being confirmed by nucleotide sequencing of the PCR products. Expression of GFP mRNA was also observed in animals tested that were positive for GFP. As well, in F1 generation animals positive for GFP and ampr sequences that were positive for GFP mRNA expression, GFP protein was detected by western blot.

Breeding F1 animals gave F2 animals that were analyzed for GFP and ampr sequences in their DNA. Animals positive for both were mated to give F3 animals. F3 animals tested positive for GFP and ampr. Multiple F3 animals tested positive via western blot for GFP protein.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for generating a transgenic animal, comprising; directly injecting a pregnant animal with DNA comprising a sequence of interest under conditions such that the pregnant animal delivers an offspring that is germ-line transgenic for said sequence of interest.

2. The method of claim 1, wherein said animal is a non-human mammal.

3. The method of claim 1, wherein said animal is a rodent.

4. The method of claim 1, wherein said injecting comprises injection of DNA into or near a vein, or other vessel.

5. The method of claim 1, wherein said injecting comprises intravenous injection into a tail vein of said pregnant animal.

6. The method of claim 1 wherein the pregnant animal is prepared for at least one injection, and wherein the DNA is administered by inserting a hollow device into or near a blood transport vessel of the pregnant animal, and introducing the DNA into or near the blood transport vessel through the hollow device so as to permit transfer of said nucleic acid material to said embryo.

7. The method to of claim 6, wherein the preparation of the pregnant animal comprises at least one step selected from the group consisting of dilating a blood transport vessel of the pregnant animal, warming the pregnant animal, immobilizing the blood transport vessel of the pregnant animal, anesthetizing the pregnant animal, sufficiently securing the pregnant animal to permit insertion a hollow device into the blood transport vessel, and having the pregnant animal immobilize the blood transport vessel, wherein the introduction of the DNA is done after implantation of the embryo into a uterine wall and prior to establishment of fetal circulation in the embryo; and wherein, the introduction of the DNA is done at an introduction pressure sufficiently low so as not to kill the pregnant animal.

8. The method of claim 1, wherein said DNA comprises a plasmid containing said sequence of interest.

9. The method of claim 1, wherein said sequence of interest encodes a marker protein.

10. The method of claim 1, wherein said sequence of interest encodes a peptide.

11. The method of claim 8, wherein said peptide is an antibody or fragment thereof

12. The method of claim 1, wherein said sequence of interest encodes a siRNA.

13. The method of claim 1, wherein said sequence of interest encodes a therapeutic protein.

14. The method of claim 1, further comprising the step of breeding said offspring to generate an F1 and subsequent generations of offspring that are transgenic for said sequence of interest.

15. The method of claim 1, further comprising the step of detecting the presence of said sequence of interest in said offspring.

16. The method of claim 1, further comprising the step of detecting the expression of said sequence of interest in said offspring.

17. The method of claim 14, further comprising the step of detecting the presence of said sequence of interest in said F1 and subsequent generations of offspring.

18. The method of claim 14, further comprising the step of detecting the expression of said sequence of interest in said F1 and subsequent generations of offspring.

19. The method of claim 1, wherein said pregnant animal is within the first 60% time period of gestation.

20. The method of claim 1, wherein said animal is a livestock animal.

21. The method of claim 1, wherein said animal is a companion animal.

22. The method of claim 1, wherein said animal is a primate.

23. A transgenic offspring generated by the method of claim 2.

24. A transgenic F1 generation animal generated by the method of claim 14.

25. A composition comprising germ-line cells of a transgenic animal generated by application of the method of claim 1.
